(19) United States
(12) Patent Application Publication
(10) Pub. No.: US 2021/0095253 A1
(43) Pub. Date: Apr. 1, 2021

(54) METHOD AND SYSTEM FOR HAIR REGROWTH USING 3D ORGANOID SYSTEM OF HAIR FOLLICLE STEM CELL

(71) Applicant: Dongha Bang, Seoul (KR)

(72) Inventor: Dongha Bang, Seoul (KR)

(21) Appl. No.: 16/981,699

(22) PCT Filed: Mar. 20, 2019
(86) PCT No.: PCT/KR2019/003258

§ 371 Date: Sep. 17, 2020

(30) Foreign Application Priority Data
Mar. 22, 2018 (KR) 10-2018-0033594

(51) Int. Cl. C12N 5/0775; C12N 5/00; C12N 5/071; C12N 5/077; A61K 35/36

(52) U.S. Cl. CPC C12N 5/0666; C12N 5/0062; A61K 9/0019; C12N 5/0656; A61K 35/36; C12N 5/0625

(57) ABSTRACT

A system and method for producing hair follicle stem cell 3D organoid using a feeder cell or cell line where the feeder cell or cell line is a dermal endothelial cell, a fibroblast cell or a cell line that is similar to the target cell or the same type of the target cell. The system and method provide rapid culture as well as a long-term sustainable 3D cell or tissue culture environment and also a treatment for hair loss.

### CROSS-REFERENCE TO RELATED APPLICATIONS

This application claims the benefit under 35 U.S.C. Section 371, of PCT International Application No. PCT/KR2019/003258, filed on Mar. 20, 2019, which claimed priority to Korean Patent Application 10-2018-0033594 filed on Mar. 22, 2018, the disclosures of which are hereby incorporated by reference herein in their entirety.

### BACKGROUND

Hair follicles are known to contain a well-characterized niche for adult stem cells including the bulge containing epithelial and melanocytic stem cells. Stem cells in the hair bulge can generate the interfollicular epidermis, hair follicle structures and sebaceous glands. The budge epithelial stem cells can also reconstitute in an artificial in vivo system to a new hair follicle. It has been known that transplanting hair follicle stem cells in balding scalp results in increase hair density and may promote new formation of hair follicles. Stem Cell Investig. 2017, 4:58. However, the result of the transplantation was somewhat limited as the amount of the transplanted stem cells was limited to the amount obtained from a patient through a physical separation. For increased effectiveness of such transplantation, a method and a system that allows culturing such stem cells in vitro in particularly in 3D. Also, after transplantation of the stem cells, adjusting planted scalp conditions for better settlement of the stem cells would help the effectiveness of such treatment.

3D cell culture techniques have led to the creation of more predictive in vitro cell models for numerous applications including cancer research, drug discovery, neuroscience and regenerative medicine. Cells naturally grow and differentiate in three dimensional environments. Cells in their natural environment have constant interactions with extracellular matrix proteins (ECM) and other cells, regulating complex biological functions like cellular migration, apoptosis, or receptor expression. Most of these interactions are lost, or significantly reduced, in traditional 2D cell cultures. Advanced 3D cell systems allow researchers to bridge the gap between classical 2D cell culture and in vivo animal models. Recently, the use of advanced 3D cell culture methods such as tumor spheroids, stem cell organoids and tissue engineering via 3D bioprinting have been implemented to more closely model real in vivo cellular responses. Improving 3D cell culture models to accurately replicate the natural environment will provide more meaningful scientific conclusions and ultimately improve human health. 3D cell culture models can be divided into two main categories: 1) scaffold-based methods using animal derived/synthetic hydrogels or structural 3D scaffolds and 2) scaffold-free approaches using freely floating cell aggregates termed spheroids.

Existing 3D methods are not without limitations, including scalability, reproducibility, sensitivity, and compatibility with high-throughput screening (HTS) instruments. Growing 3D cell models take long time to be utilize as short-term clinical test uses. Also, maintaining 3D cell models in vitro for a long period of time is challenging. Accordingly, a new method and system for 3D cell culture is needed. A 3D cell model which can be cultured in a short period of time can be used for a patient specific medical treatment and can improve efficiency of drug discovery.

The disclosed embodiments of the present disclosure are directed to overcoming one or more of the problems with existing 3D culture methods and systems and illustrate the present invention. The scope of the invention shall not be limited to the disclosed embodiment.

### SUMMARY OF THE DISCLOSURE

One embodiment of the present invention relates to a method for producing 3D cell mass efficiently and effectively using a secondary cell line to support the rapid growth of a hair follicle stem cell to be grown to a three-dimensional structure or three dimensional mass of cells, e.g. organoid, which is herein defined as any 3D mass of cells or 3D cell models including conventional organoid and spheroid as well as tissue. Such 3D cell models have many benefits over conventional two-dimension cell structures. For example, 3D organoid is physiologically more like actual biologic organ or tissue and can be used for drug discovery or screen or medical treatments such as cell therapies, etc. 3D organoid can be used for creating micro physiological systems or organs-on-chips to study difficult clinical problems and development of potential treatments. These human surrogates make use of pumpless self-contained systems that are robust, easy to use, and low cost and allow measurement of metabolic and functional responses.

In particular, one embodiment provides a method for culturing hair follicle stem cells using patient derived hair follicle stem cells cultured by using a feeder cell. Where the feeder cell in a medium may be provided in a way that the feeder cell medium is separated from the medium for hair follicle stem cells. A medium with various growth factor may be supplied on the top of the feeder cell medium and the hair follicle stem cell medium. The resulting culture bed is incubated

The feeder cell may be a dermal endothelial cell or a fibroblast cell, preferably a dermal endothelial cell from the patient. Scalp tissue may be separated from a patient. Using a single cell isolation method, dermal endothelial cells and hair follicle stem cells can be obtained from the scalp tissue. The dermal endothelial cells may be further cultured to expand its population such as 3d culture before using the dermal endothelial cells as the feeder cell.

Another embodiment provides a method for growing a 3D organoid of hair follicle stem cells by preparing a first medium with a follicle stem cell from a patient, preparing a second medium with a dermal endothelial cell from the patient, placing the first medium and the second medium in a grow substrate, placing a conditioned medium over the growing substrate to cover the first medium and the second medium, resulting in a culture plating, incubating the culture plating to grow the 3D organoid and harvesting cultured hair follicle stem cells.

Another embodiment provides a method for promoting hair growth in a patient by injecting hair follicle stems cells derived from the patent into the patient's skin where the hair follicle stems cells are harvested from a culture bed comprising a hair follicle stem and a dermal endothelial cell from the patient wherein the dermal endothelial cell is a feeder cell. In order to improve the hair growth, dermal endothelial cells may be injected along with the hair follicle stem cells.

The target cell may be placed on a separable substrate plate so that the substrate plate having the target cell can be transferred to a different culture bed. In that way, the cultured target cell can be exposed a new bed with a flash second cell or cell line. The target cell may be any cell from any part of human body such as an organ tissue or skin tissue but may be from a foreign cell such as cancer cell.

The culture substrate is a glass bottom dish where a portion of the dish bottom has a glass plate, which can be removed from the glass bottom dish. For example, the portion of the matrigel mixture over the medium containing the target cell may be removed conveniently by lifting the glass plate and relocated into another glass bottom dish to provide a fresh feeder cell. The materials for the glass plate and the glass bottom dish can vary as needed. For example, plastic material may be used.

### BRIEF DESCRIPTION OF THE DRAWINGS

FIG. 1 is an illustration of an embodiment according to the present disclosure.

FIG. 2. (a) is a side view of an illustration of an embodiment according to the present disclosure and (b) is another side view of an embodiment with a removable plate.

FIG. 3 is an illustrative flow chart utilizing an embodiment according to the present disclosure.

FIG. 4 is an illustrative flow chart utilizing an embodiment according to the present disclosure.

FIG. 5 is an illustrative flow chart utilizing an embodiment according to the present disclosure.

FIG. 6 is an illustrative flow chart utilizing an embodiment according to the present disclosure.

FIG. 7 is an illustration showing isolating hair follicle stem cells and dermal endothelial cells from hair tissues.

FIG. 8 is an illustration of a process showing culture system for growing 3D organoid of isolated hair follicle stem cells using the dermal endothelial cells as feeder cells.

FIG. 9 is an illustration of transplantation of cultured hair follicle stem cells for regrowth of hair.

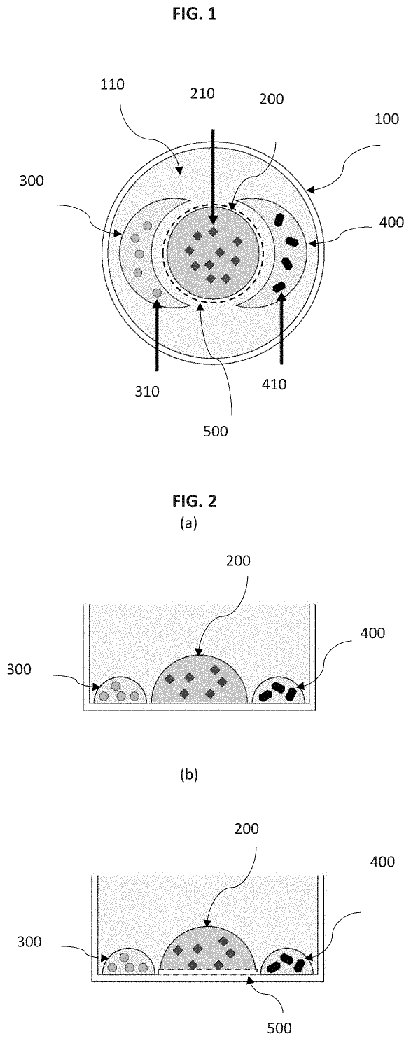

### DETAILED DESCRIPTION OF PREFERRED EMBODIMENTS

FIG. 1 illustrates a culture system according to one embodiment of the present invention. Target cells, such as a cancer cell from a patient, are obtained from a patient and separated into a small piece. The separated cells 210 are placed in a conditioned medium such as a microfluidic medium and in matrigel. The mixture 200 is placed in a glass bottom dish 100 and incubated to solidify the mixture on the dish. A feeder cell or cell line 310 in a conditioned medium and a matrigel is provided in the same dish but can be placed in a way that the feeder cell or cell line mixture 300 is separated from the target cell mixture 200. The feeder cell or cell line is not necessary from the patient but from a third party. The separation allows harvesting cultured target cells without any contamination of cells from the feeder mixture.

The feeder cell or cell line is to promote the growth of the target cell and is not for own growth. The feeder cell or cell line is preferably an endothelial cell or fibroblast cell. More preferably, the endothelial cell is a human dermal microvascular endothelial cell. Even more preferably, the feeder cell or cell line is an endothelial cell or fibroblast cell from an organ that is similar to or the same as the organ from which the target cell is obtained.

The culture bed may have a second feeder cell or cell line 410 which is similarly prepared in a conditioned medium and matrigel. The second feeder cell or cell line mixture 400 may also be placed in a way that the mixture 400 is separated from the target cell or cell line mixture. The second feeder cell or cell line can be placed in the dish. The second feeder cell or cell line is preferably cell or cell lines that is similar to or the same cell or cell line as the target cell. Since the target cell mixture bed is separated from the feeder cell beds, cultured target cells can be harvested with any contamination from the feeder cell beds.

Optionally, the mixture 200 is placed on a separate removable plate 500, which allows removing the mixture bed from the dish and relocate to another dish to provide additional feeder cells or nutrients.

After the target cell and feeder cell mixtures are placed in the dish, a conditioned medium can be filled in the dish.

The conditioned medium can be chosen from various media. Many cell culture medium formulations are documented in the literature and a number of media are commercially available. Once the culture medium is incubated with cells, it is known to those skilled in the art as “spent” or “conditioned medium”. Conditioned medium contains many of the original components of the medium, as well as a variety of cellular metabolites and secreted proteins, including, for example, biologically active growth factors, inflammatory mediators and other extracellular proteins.

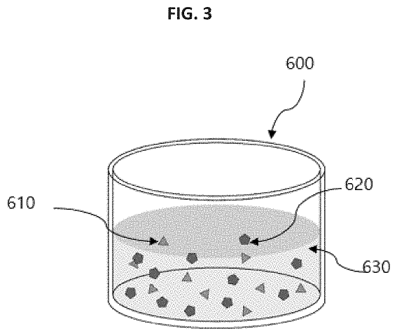

When the cultured target cells do not require to be separated from foreign cells from the feeder cells, the feeder cell or cell line mixtures 610 may be mixed with the target cell mixture 620 in a conditioned media 630 as the illustrated culture bed 600 in FIG. 3.

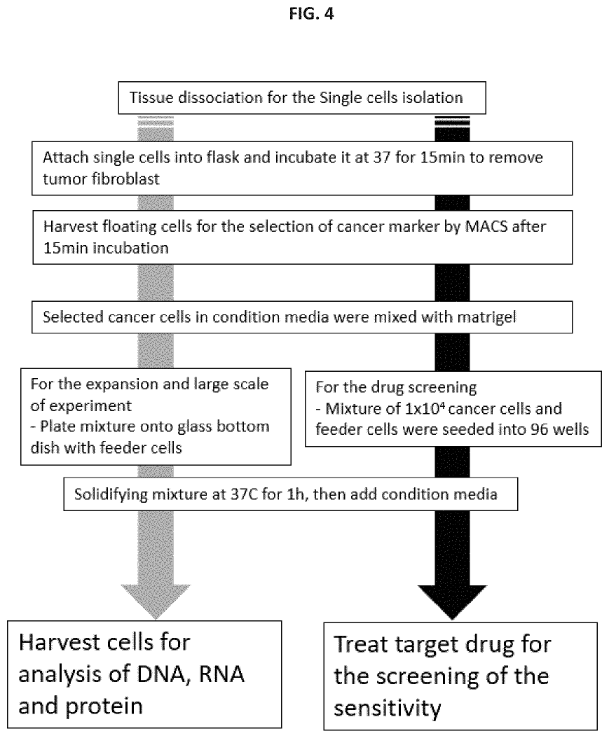

FIG. 4 illustrates example protocols for 3D culture and uses of cultured cells. Tissue from a patent is dissociated for a single cell isolation. The single cells are seeded into a flask and the attached flask is incubated at 37° C. for 15 minutes to eliminate tumor fibroblast. After this step, floating cells are harvested for cancer marker selection through MACS® technology (https://www.miltenyibiotec.com/DK-en/products/macs-cell-separation.html. Selected cancer cells in a conditioned medium are mixed with matrigel. For an expansion and large scale of experiment, the matrigel mixture is plated on a glass bottom dish with feeder cells as described above. For a drug screening, a matrigel mixture with 1×10⁴ cancer cells and feeder cells is plated into 96 wells. Then, the mixture may be solidified at 37° C. for 1 hr., and a conditioned medium is added on the top of the solidified mixture.

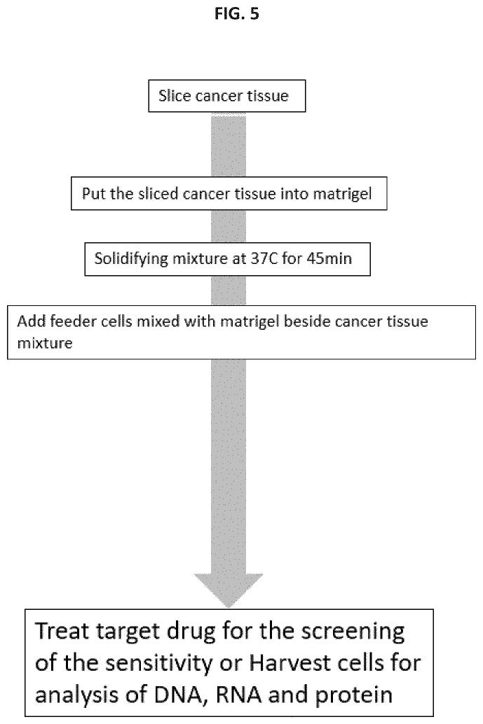

The 3D culture method and system of the present invention can be used to culture tissues. For example, FIG. 5 illustrates a method for 3D culture of tissue. A tissue sample is obtained by slicing tissue from a patient. The tissue sample may be placed in matrigel, of the mixture may be solidified at 37° C. for 45 minutes. Then, feeder cells mixed with matrigel are placed as described herein for 3D culture. The cultured tissue can be used for drug screening or sensitivity tests or cell harvesting for other tests or uses.

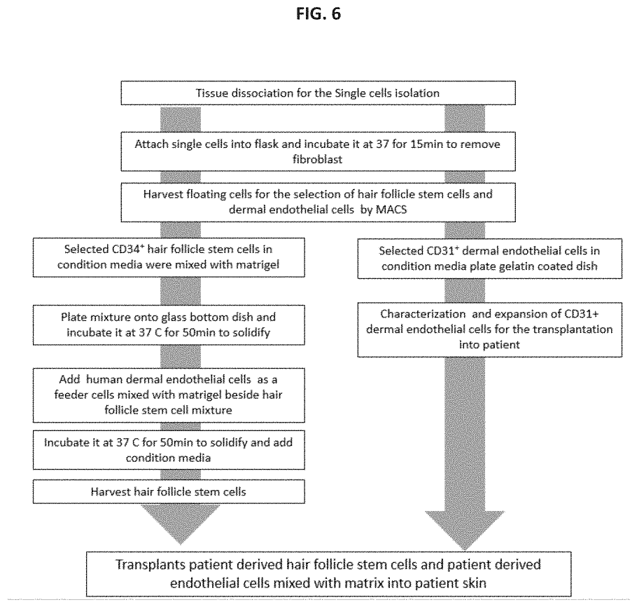

An embodiment of the present invention can be used to culture cells or tissues for direct transplantation to a patient. For example, FIG. 6 illustrates an example protocol for culturing hair follicle stem cells and dermal endothelial cells and transplanting them to a patient's skin. In this case, the target cell and the feeder cell can be obtained from a patent to whom cultured cells are to be transplanted. A hair tissue sample from a patient is obtained and the hair tissue sample is subject to the single cell isolation process and using cell separation technique such as MACS, hair follicle stem cells and dermal endothelial cells are respectively harvested. the dermal endothelial cells are cultured to increase cell mass, which can be used as a feeder for the hair follicle stem cell culture as well as to be transplanted to the patient along with the cultured hair follicle stem cells to regrow hair in the patient.

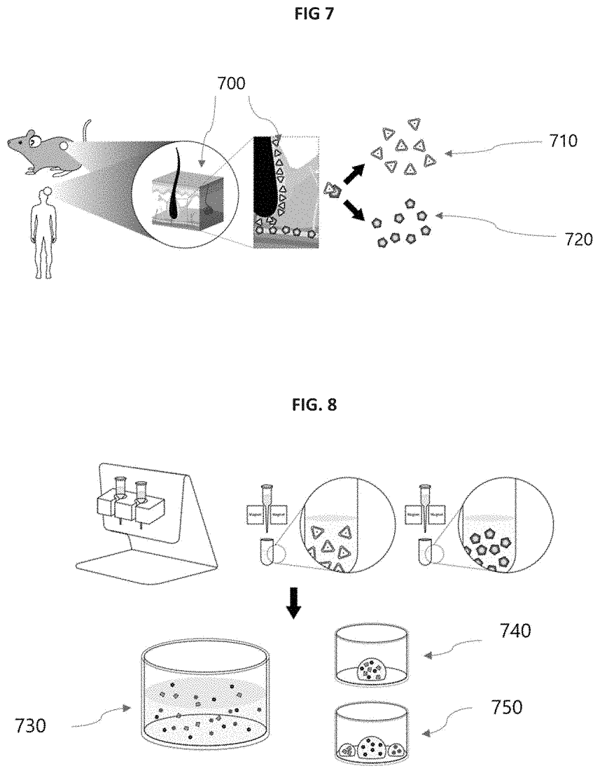

FIG. 7 illustrates that hair tissue 700 is obtained from a patient through skin biopsy. Hair follicle stem cells and dermal endothelial cells are isolated from the tissue using single cell isolation techniques known in the art. Hair tissue

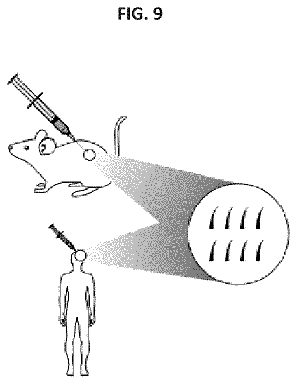

FIG. 8 is an illustration of a process showing culture system for growing 3D organoid of isolated hair follicle stem cells using the dermal endothelial cells as feeder cells.

FIG. 9 is an illustration of transplantation of cultured hair follicle stem cells for regrowth of hair.

The various stem cell colonies grown in 3 D culture can be used in stem cell treatment, drug discovery, drug sensitivity test, and other stem cell science. In particularly, the 3D culture methods and systems according to the present invention allow grow stem cell colonies or tissue in a shorter period of time than the conventional methods and systems. The speed is particularly important to utilize the method and system as patient-specific medicine because, for example, a cancer patient would require a quick drug screen or cell therapy as cancers usually grow very rapidly.

Also, the 3D culture systems and methods according to the present invention sustain the cultured cells or tissue for a long period of time. If necessary, the cultured cells o tissues can live several months, even longer. This is a very important feature for drug discovery or tests.

The following examples are illustrative purpose only. The scope of the invention should not be limited to these examples.

### Example 1. Hair Follicle Stem Cell Isolation

a. Anesthetize skin with lidocaine crime before skin biopsy
b. Collect mouse or human skin including dermis by punch biopsy (1 cm diameter)
c. Put the skin tissue into 50 ml conical tube filled with washing medium
d. High glucose DMEM with 1% FBS
e. Transfer the skin tissue onto a dish
f. Mincing skin tissue with scissor as small as possible and put it into dissociation solution
g. Dissociation solution: collagenase type II/DNase I in HBSS
h. Incubate minced tumor tissue for an hour at 37° C.
i. Add an equal volume of FBS and strain the solution through 100 um strainer
j. Spin filtered single cells with 1000 rpm for 10 min
k. Wash cell pellet with PBS and spindown at 1000 rpm for 5 min (3 times)
l. If you can find RBC in the pellet, use RBC lysis buffer as following commercial instruction.
m. The cells were then resuspended in 400 ul HBSS/0.5% BSA, mixed with 1 ul CD34-mibrobead mAb (1:200), and rotated for 30˜40 min at 4° C.
n. The cells were then washed once with sterile MACS buffer at 1200 rpm for 5 min in microcentrifuge.
o. Before application of the cells to the magnetic MACS columns (MS columns), each column was equilibrated with 1 ml MACS buffer. 1 ml MACS buffer was added to the cells and the entire volume was applied to the column.
p. The column was washed three times with 500 ul MACS buffer and gentle positive pressure was applied with plunge before the last wash.
q. The columns were then removed from the magnet the cells were eluted with 1 ml MACS buffer with positive pressure into 15 ml conical tube.
r. The cells were then centrifuged at 1000 rpm for 5 min and resuspended with hair follicle stem cell growth media

Dermal Endothelial Cells Isolation
a. The cells were then resuspended in 400 ul HBSS/0.5% BSA, mixed with 1 ul CD31-mibrobead mAb (1:300), and rotated for 30˜40 min at 4° C.

3D Organoid Culture of Hair Follicle Stem Cells
a. Mixed with isolated hair follicle stem cells with dermal endothelial cells in Matrix (Matrigel, BD) which is solidifying at 37° C. and liquidation at 4° C.
b. Plate the mixture on the center of glass-bottom dish and add hair follicle stem cell condition media after the mixture solidified at 37° C. with 5% CO2 in a humidified chamber for 1 hour.
c. Change the medium every 3-4 days.
d. Passing hair follicle organoid for 7 days after plating.

What is claimed is:

1. Method for culturing hair follicle stem cells, comprising
providing a first medium comprising a row hair follicle stem cell from a patient on a growing substrate and
providing a second medium comprising a feeder cell on the growing substrate,
wherein the second medium is placed in a way that the second medium is physically separated from the first medium to product the 3D organoid.

2. The method according to claim 1, wherein the second cell in the second medium is a dermal endothelial cell or a fibroblast cell.

3. The method according to claim 1, wherein the row hair follicle stem cell is from a scalp tissue of a patient and the second cell is an endothelial cell from the scalp tissue.

4. The method according to claim 3, wherein the second cell is an endothelial cell, which has been subject to a 3D culture.

5. A method for growing a 3D organoid of hair follicle stem cells, comprising
a) preparing a first medium comprising a follicle stem cell from a patient;
b) preparing a second medium comprising a dermal endothelial cell from the patient
c) placing the first medium and the second medium in a grow substrate;
d) placing a conditioned medium over the growing substrate to cover the first medium and the second medium, resulting in a culture plating;
e) incubating the culture plating to grow the 3D organoid; and
f) harvesting cultured hair follicle stem cells

6. A method for promoting hair growth in a patient, comprising injecting hair follicle stems cells derived from the patent into the patient's skin where the hair follicle stems cells are harvested from a culture bed comprising a hair follicle stem and a dermal endothelial cell from the patient wherein the dermal endothelial cell is a feeder cell.

7. The method according to claim 6, further comprising injecting dermal endothelial cells from the patient.

8. The method according to claim 6, wherein the dermal endothelial cells are harvested from a culture bed.
